(19) United States
(12) Patent Application Publication
(10) Pub. No.: US 2018/0289550 A1
(43) Pub. Date: Oct. 11, 2018

(54) SYSTEMS, APPARATUSES, KITS AND METHODS FOR IMPROVED MEDICAL PROCEDURES

(71) Applicant: Advent Access Pte. Ltd., Singapore (SG)

(72) Inventors: Hongye Ye, Singapore (SG); Ruey Feng Peh, Singapore (SG); John Shulze, Singapore (SG)

(73) Assignee: Advent Access Pte. Ltd., Singapore (SG)

(21) Appl. No.: 15/945,650

(22) Filed: Apr. 4, 2018

Related U.S. Application Data
(60) Provisional application No. 62/481,123, filed on Apr. 4, 2017.
(60) Provisional application No. 62/515,946, filed on Jun. 6, 2017.

(51) Int. Cl. A61F 13/00; A61F 13/42; A61M 25/06; A61M 1/14; A61M 1/36

(52) U.S. Cl. CPC A61F 13/00; A61F 2013/00817; A61F 13/00059; A61F 13/00063; A61F 13/42; A61M 25/0637; A61M 1/14; A61M 1/3661; A61F 2013/00417; A61F 2013/00425; A61F 2013/00434; A61F 2013/00438; A61F 2013/00468; A61F 2013/00663; A61F 2013/424; A61F 13/00055

(57) ABSTRACT

Methods, materials, articles, assemblies, systems, devices, kits and computer hardware/software for improving medical procedures, including, but not limited to, hemodialysis. Particular beneficial designs and uses of disinfecting patches, scab removal patches, hemostatic patches, healing patches and artificial scabs are included. For example, scab removal patches that avoid tenting, hemostatic patches with needle stabilizing elements, hemostatic patches applying pressure at a skin puncture site and a vessel puncture site, artificial scabs that release antimicrobial agents, etc.

### RELATED APPLICATION(S)

This application claims priority to and hereby incorporates by reference the contents of U.S. Provisional Application No. 62/481,123, filed Apr. 4, 2017, and U.S. Provisional Application No. 62/515,946, filed Jun. 6, 2017.

### BACKGROUND

Hemodialysis is a process for purifying the blood of a person whose kidneys are unable to do so sufficiently on their own. The process is typically conducted in an outpatient facility staffed by specialized nurses or technicians and requires a patient to undergo three to four hour treatments at the facility, typically three times per week. During the procedure, a patient's blood is drawn out from a vascular access site (typically in the patient's arm), run through a dialysis machine, and then pumped back into the patient.

Aside from the blood purification process itself, there are many other aspects of the process that a patient undergoes during each dialysis session. For example, the patient's vascular access sites must be disinfected, scabs resulting from prior access may need to be removed, blood vessels must be accessed (e.g., through needling), hemostasis (the stoppage of bleeding) needs to occur after the procedure, etc.

Aspects of the present disclosure are directed primarily to methods, materials, articles, assemblies, systems, devices, kits and computer hardware/software for improving hemodialysis. However, it is understood and contemplated that certain technologies disclosed herein may be utilized in medical procedures more generally and are thus not intended to be limited to use only in hemodialysis. As one example, the hemostasis patches disclosed herein may be utilized to facilitate hemostasis for wounds other than hemodialysis needling sites.

### SUMMARY

Methods, materials, articles, assemblies, systems, devices, kits and computer hardware/software for improving medical procedures, including, but not limited to, hemodialysis are disclosed. Also included are particular beneficial designs and uses of disinfecting patches, scab removal patches, hemostatic patches, healing patches and artificial scabs. For example, scab removal patches that avoid tenting, hemostatic patches with needle stabilizing elements, hemostatic patches applying pressure at a skin puncture site and a vessel puncture site, artificial scabs that release antimicrobial agents, etc.

In one aspect of the present disclosure, a hemostatic patch includes a support layer with a patient adhesive and a sponge. The support layer may also include a vessel-focused hemostatic material located away from the sponge that can be configured to apply a hemostatic force above da blood vessel, at the location where a needle has punctured the blood vessel. In some implementations, the distance between the sponge and the vessel-focused hemostatic material can be configured to be an expected distance between a needle/skin entry point and a needle/vessel entry point in a hemodialysis procedure.

Additional hemostatic patch designs may include an above-needle portion and a below-needle portion, where the below needle portion may include a needle stabilizing element that may secure the needle in place during a hemodialysis procedure. A wedge may also be placed above or adjacent to the below-needle portion in order to further support a needle and may be configured to have a top surface with an angle corresponding to an expected needle angle.

In other hemostatic patch designs, a sponge on the patch can be configured to expand upon absorption of a fluid, while the support layer of the patch is designed to be substantially inelastic, such that a hemostatic force will be exerted upon the sponge's absorption of a fluid.

Also disclosed herein are exemplary disinfecting patches that can include a patient adhesive and an antiseptic on a support layer that may also include a handle portion to allow for one-handed removal of the patch. In some variations, the antiseptic portion of the disinfecting patch can include an antiseptic configured to provide sufficient disinfection for a hemodialysis needling procedure after 30 or 60 seconds of wearing by a patient.

Also disclosed herein are exemplary scab removal patches that may include a support layer, a patient adhesive, and a scab removal portion with an adhesive configured to adhere to a scab. The adhesive may be configured to provide maximum contact between the scab adhesive and the scab, and also to soften the scab, and may thus facilitate easy and complete removal of a scab from a patient.

Some implementations of the present disclosure include the scab removal patch being removably connected to a disinfecting patch, for example, by a perforation.

The present disclosure also includes designs of wound closure apparatuses or “artificial scabs” that can have a head portion constructed from a first material and a rod portion that may be constructed from a second material, different from the first material. In addition, the head may have a partially porous wound-facing portion to facilitate formation of a natural scab adhering the wound closure apparatus to or near a wound.

Implementations of the current subject matter, especially with regard to sensors that may be included on the patches disclosed herein, can include, but are not limited to, methods consistent with the descriptions provided herein as well as articles that comprise a tangibly embodied machine-readable medium operable to cause one or more machines (e.g., computers, microprocessors, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating perspective views of an exemplary disinfecting patch and an exemplary scab removal patch in accordance with certain aspects of the present disclosure.

FIG. 2 is a diagram illustrating use of an exemplary disinfecting patch in accordance with certain aspects of the present disclosure.

FIG. 3 is a diagram illustrating uses of an exemplary scab removal patch in accordance with certain aspects of the present disclosure.

FIG. 4 is a diagram illustrating use of an exemplary scab removal patch in accordance with certain aspects of the present disclosure.

FIG. 5A is a diagram illustrating an exemplary hemostatic patch in accordance with certain aspects of the present disclosure.

FIG. 5B is a diagram illustrating an exemplary hemostatic patch having an above-needle portion and a below-needle portion in accordance with certain aspects of the present disclosure.

FIG. 6 is a diagram illustrating an exemplary use of an exemplary hemostatic patch in accordance with certain aspects of the present disclosure.

FIG. 7 is a diagram illustrating an exemplary hemostatic patch and withdrawal of a needle in accordance with certain aspects of the present disclosure.

FIG. 8 is a diagram illustrating the use of an exemplary hemostatic patch in accordance with certain aspects of the present disclosure.

FIG. 9 is a diagram illustrating hooks as an exemplary needle stabilizing element in accordance with certain aspects of the present disclosure.

FIG. 10A is a diagram illustrating another exemplary needle stabilizing element in accordance with certain aspects of the present disclosure.

FIG. 10B is a diagram illustrating another exemplary needle stabilizing element in accordance with certain aspects of the present disclosure.

FIG. 11A is a diagram illustrating a side elevation of exemplary wedge and a hemostatic patch in accordance with certain aspects of the present disclosure.

FIG. 11B is a diagram illustrating a perspective view of an exemplary wedge with its butterfly cutout and a hemostatic patch in accordance with certain aspects of the present disclosure.

FIG. 12A is a diagram illustrating a side elevation of an exemplary wedge and a hemostatic patch in accordance with certain aspects of the present disclosure.

FIG. 12B is a diagram illustrating a perspective view of an exemplary wedge with its butterfly cutout and a hemostatic patch in accordance with certain aspects of the present disclosure.

FIG. 13A is a diagram illustrating a top view of an exemplary healing patch in accordance with certain aspects of the present disclosure.

FIG. 13B is a diagram illustrating a bottom view of an exemplary healing patch in accordance with certain aspects of the present disclosure.

FIG. 14 is a diagram illustrating an exemplary artificial scab in accordance with certain aspects of the present disclosure.

FIG. 15 is a diagram illustrating an exemplary artificial scab engaging a subcutaneous needle guiding device in accordance with certain aspects of the present disclosure.

FIG. 16 is a diagram illustrating an exemplary artificial scab constructed of two different materials in accordance with certain aspects of the present disclosure.

FIG. 17 is a diagram illustrating an exemplary artificial scab including a porous structure in accordance with certain aspects of the present disclosure.

FIG. 18 is a diagram illustrating an exemplary method of use of some of the technologies of the present disclosure.

FIG. 19 is a diagram illustrating an exemplary a kit containing some of the technologies of the present disclosure.

### DETAILED DESCRIPTION

The present disclosure is directed to methods, materials, articles, assemblies, systems, devices, kits and computer hardware/software for improving medical procedures, including, but not limited to, hemodialysis.

Implementations of the technologies disclosed herein may prove particularly advantageous for various facets of procedures taking place during a hemodialysis session. For example, disclosed herein are methods and apparatuses for facilitating disinfection of points of vascular access, including particular designs for a disinfecting patch.

In addition, disclosed herein are technologies relating to the removal of scabs that may exist at needling sites, which must be removed prior to commencement of dialysis at the same sites (as may occur when utilizing the buttonhole technique for hemodialysis).

Following hemodialysis, hemostasis must be achieved. Technologies are disclosed herein to facilitate such, including particular hemostatic patch designs that, among other things, can be configured to apply passive compressive forces at a needling site and also above the site where the needle enters the blood vessel.

Also disclosed herein are technologies to optimize needle site healing and, optionally, to provide an artificial scab to seal a needling tract without formation of a natural scab.

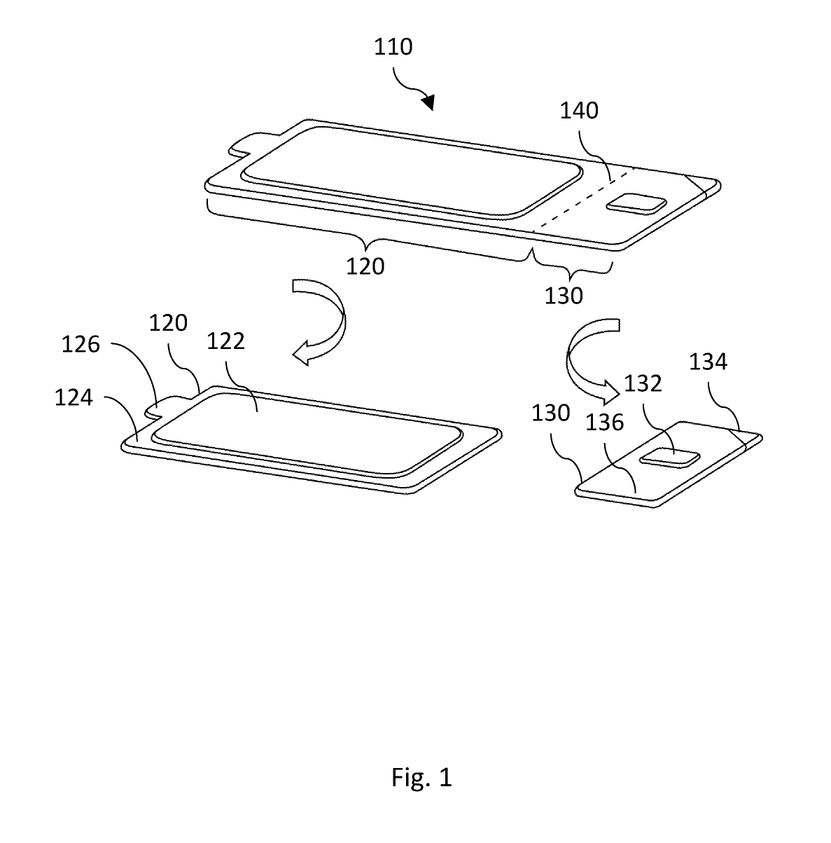

FIG. 1 is a diagram illustrating perspective views of an exemplary disinfecting patch 120 and an exemplary scab removal patch 130 in accordance with certain aspects of the present disclosure.

Disinfecting patch 120 and scab removal patch 130 may be manufactured as a single patch assembly 110 or may be manufactured separately. In the case where the patches are combined, disinfecting patch 120 may be removably connected to scab removal patch 130 by, for example, a perforation 140, or by a thin connecting section, so as to facilitate separation.

In some implementations, a removable cover can be affixed to disinfecting patch 120 and/or scab removal patch 130. The removable cover can be a thin layer of plastic, metal foil, or other material that can be peeled, slid, rolled, or otherwise removed from disinfecting patch 120 and/or scab removal patch 130.

An exemplary disinfecting patch 120, as shown in FIG. 1, may include a support layer 124 and an antiseptic portion 122 included on at least a portion of support layer 124. The support layer 124 may be made of, for example, a single material or a blend of materials, such as cotton, nylon, spandex, etc. The layer may optionally be made waterproof or water resistant.

The antiseptic portion 122 of disinfecting patch 120 may be made, for example, of a single material or a blend of natural and/or synthetic absorbent material(s), such as cotton, cellulose, hydrogels, etc. Antiseptic portion 122 may be configured to be wet with an antiseptic, for example, carrying a mobile liquid or a liquid trapped in a hydrogel matrix. This liquid may also help to soak and soften any underlying scab that may be present.

Antiseptic agents carried or released by disinfecting patch 120 can include one or more of the following classes of compounds: alcohols (e.g. ethanol, isopropanol), aldehydes (e.g. glutaraldehyde, formaldehyde), anilides (e.g. triclocarban), biguanides (e.g. chlorhexidine, alexidine), bisphenols (e.g. triclosan, hexachlorophene), diamidines (e.g. propamidines, dibromopropamidines), halogen-releasing agents (e.g. chlorine compounds, iodine compounds), halophenols (e.g. chloroxylenol), heavy metal derivatives (e.g. silver compounds, mercury compounds), peroxygens (e.g. hydrogen peroxide, ozone, peracetic acid), phenols and cresols (e.g. phenol, cresol) and quaternary ammonium compounds (e.g. cetrimide, benzalkonium chloride).

In particular implementations, the disinfecting patch's antiseptic portion 122 can include an antiseptic configured to provide sufficient disinfection for a hemodialysis needling procedure after 15 seconds of wearing by a patient, after 30 seconds of wearing by patient, or after 60 seconds of wearing by patient, depending upon the configuration of the particular antiseptic.

The support layer 124 of disinfecting patch 120 may also include a patient adhesive on at least a portion of the support layer to facilitate the patch staying in place so that antiseptic portion 122 can contact the skin for a specified period of time. In one implementation, the patient adhesive may be a thin layer of hypoallergenic adhesive included generally around the periphery of support layer 124. As used herein, the “periphery” can include a whole or partial outline of a region and need not only be precisely at the edge of the region.

The disinfecting patch 120 can be formed in essentially any shape and size, including one large enough to cover the entire working area of skin around a needling site. Representative designs can include a rectangular pad with a length in the range of 1 cm to 15 cm and a width of 1 cm to 8 cm, a circular pad having a diameter in the range of 1 cm to 10 cm, or an oval patch with a long diameter range of 1 cm to 15 cm and a short diameter range of 1 cm to 8 cm. Depending on embodiment details, the disinfecting patch 120 may cover an area ranging from 1 cm² to 94 cm².

In some implementations, disinfecting patch 120 may include a feature such as a handle portion 126 that can facilitate one-handed removal of the patch. In one example, disinfecting patch 120 may be designed to have an irregular shape where handle portion 126 may include a protruding portion of the support layer 124, as shown in FIG. 1. In another variation, the handle portion may simply be a peripheral portion of support layer 124 (for example, a corner or a protruding portion) that is devoid of patient adhesive, so as to facilitate easy removal with one hand.

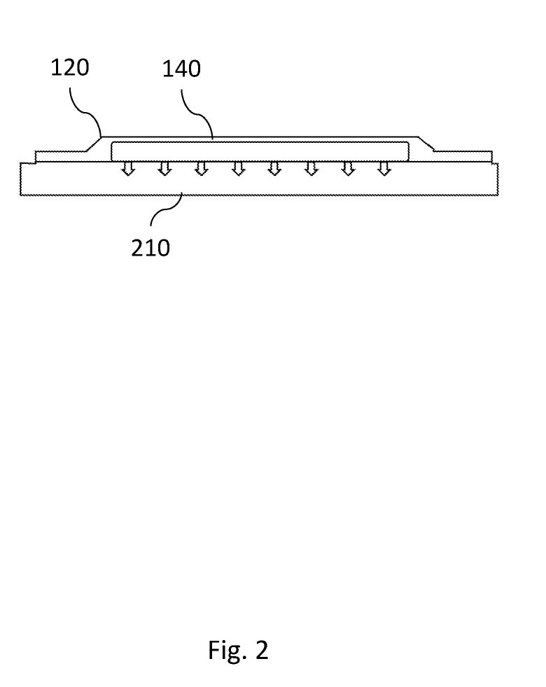

FIG. 2 is a diagram illustrating an exemplary use of disinfecting patch 120 in accordance with certain aspects of the present disclosure. Disinfecting patch 120 can be designed for adhering to a patient's skin 210 (e.g., at a planned skin puncture site), for example, through placement by a patient with one hand. Disinfecting patch 120 can be left on the patient's skin for a particular duration, which may range, for example, from 15 seconds to 15 minutes. After the intended duration, disinfecting patch 120 can be peeled off by the patient with one hand and discarded.

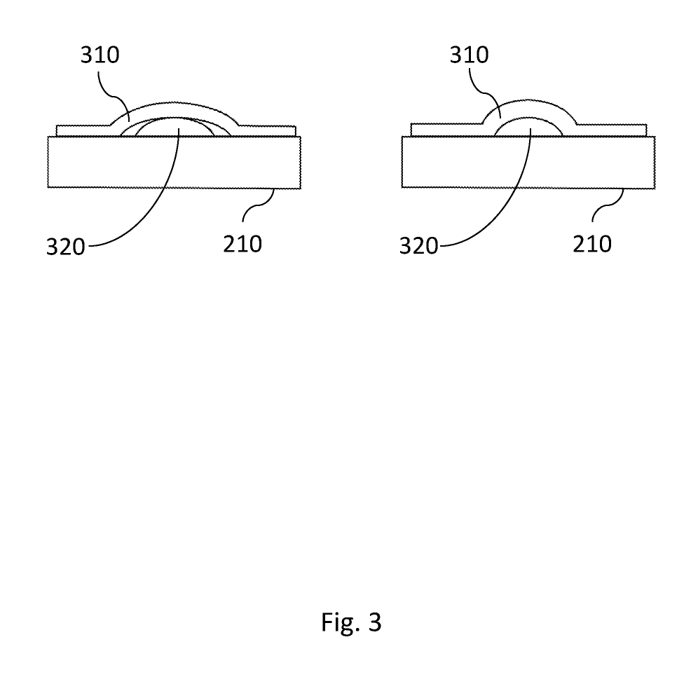

Scab removal patch 130 (as shown in FIG. 1) can include a support layer 136 and a hypoallergenic patient adhesive included on at least a portion of support layer 136 in order to adhere the patch to a patient, for example, for a scab removal procedure prior to a hemodialysis session. Scab removal patch 130 can include a scab removal portion 132 incorporating a scab adhesive 310, as shown in FIG. 3. Through the scab adhesive, scab removal patch 130 can be configured to remove a scab from a patient when the patch itself is removed from the patient.

The scab removal patch 130 can carry or contain one or more types of scab adhesive(s) 310 including, but not limited to, quaternized polymers from one or more of the following cationic monomers: imethylaminoethyl acrylate, dimethylaminoethyl methacrylate, dimethylaminopropyl acrylamide, dimethylaminopropyl methacrylamide, dimethylaminostyrene, dimethylaminomethylstyrene, 4-vinyl pyridine, 2-vinyl pyridine, or copolymers from the above-mentioned cationic monomers and one or more of the following amphoteric monomers: N-(3-sulfopropyl)-N-acryloyloxyethyl-N,N-dimethylammonium betaine, N-(3-sulfopropyl)-N-methacroylamidepropyl-N,N-dimethylammonium betaine, N-(3-carboxymethyl)-N-methacroylamidepropyl-N,N-dimethylammonium betaine, and N-carboxymethyl-N-methacroyloxyethyl-N,N-dimethylammonium betaine, or copolymer from the above mentioned cationic monomers and one or more of the following non-salt-forming monomers from the following groups: vinyl esters of aliphatic carboxylic acid, (meth)acrylic esters, alkyl vinyl esters, N-vinyl cyclic amides, styrene and alkyl-substituted styrene. The scab adhesive may also contain natural polymers such as sodium alginate, gelatin, corn starch, hydroxypropyl methylcellulose and so on.

Scab removal portion 132 and the included scab adhesive(s) 310 can be configured to provide maximum contact between scab adhesive 310 and scab 320, as shown in the right-hand side of FIG. 3. In this way, “tenting” between scab adhesive 310 and scab 320 (as shown in the left hand side of FIG. 3), can be avoided. As used herein, “tenting” refers to incomplete contact between a scab adhesive and a scab. The scab adhesives, as contemplated herein, thus result in improved conformity to the shape and size of a scab and provide a firmer ‘grip’ on the scab. This leads to easier and more complete removal of scab 320 upon removal of the scab removal patch from the skin surface.

Scab removal portion 132 may also be configured to soften scab 320, for example, through utilization of water or other fluids contained in scab removal portion 132. Softening of scab 320 can also facilitate easier and more complete removal of the scab when patch 130 is peeled off of the skin surface.

Scab removal patch 130 can be made in essentially any shape and size, but can be large enough to cover the area around scab 310. One implementation of scab removal patch 130 can be, for example, a rectangular patch with a length in the range of 1 cm to 15 cm, a width of 1 cm to 8 cm; a circular patch with a diameter in the range of 1 cm to 10 cm, or an oval patch with a long diameter range of 1 cm to 15 cm and a short diameter range of 1 cm to 8 cm. Scab removal patch 130 can cover an area ranging from 1 cm² to 94 cm². Support layer 136 may optionally be waterproof or water resistant and can be made of a single material or blend of materials such as cotton, nylon, spandex, etc. Scab removal portion 132 can be made of a single material or a blend of natural or synthetic absorbent material(s), such as cotton, cellulose, hydrogels, etc. Scab removal portion 132 can be wet with sterile saline or water and with scab adhesive(s) carried therein.

In some implementations, scab removal patch 130 may include a feature such as handle portion 134 that can facilitate one-handed use in removal of the patch. In one example, scab removal patch 130 may be designed to have an irregular shape where handle portion 134 may comprise a protruding portion of the support layer. In another variation, handle portion 134 may simply be a peripheral portion of the support layer (for example, a corner or a protruding portion) that is devoid of patient adhesive so as to facilitate easy removal with one hand, as depicted in FIG. 1 as element 134.

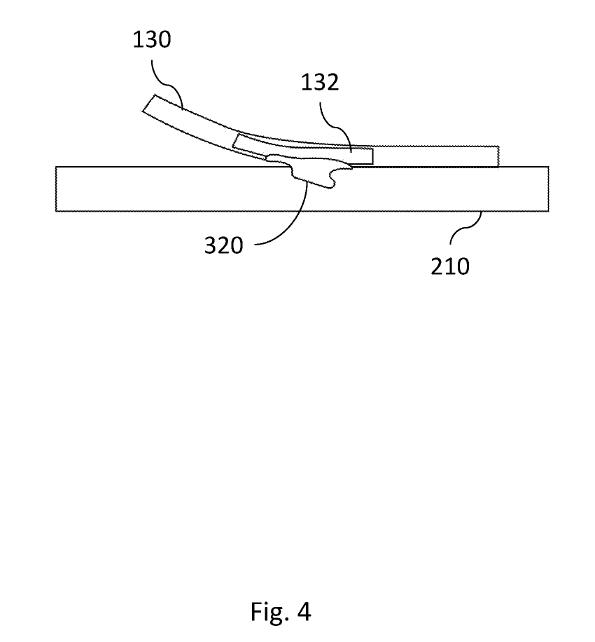

FIG. 4 is a diagram illustrating use of an exemplary scab removal patch 130 in accordance with certain aspects of the present disclosure. Scab removal patch 130 may be applied over scab 320 and can be left on a patient's skin for several minutes, e.g., 10 minutes. During this time, scab removal patch 130 will adhere to scab 320, and optionally soften the scab as well. Scab removal patch 130 can then be peeled off by a caregiver or the patient, with one hand, at which time scab 320 will remain adhered to the patch and will be removed along with the patch itself.

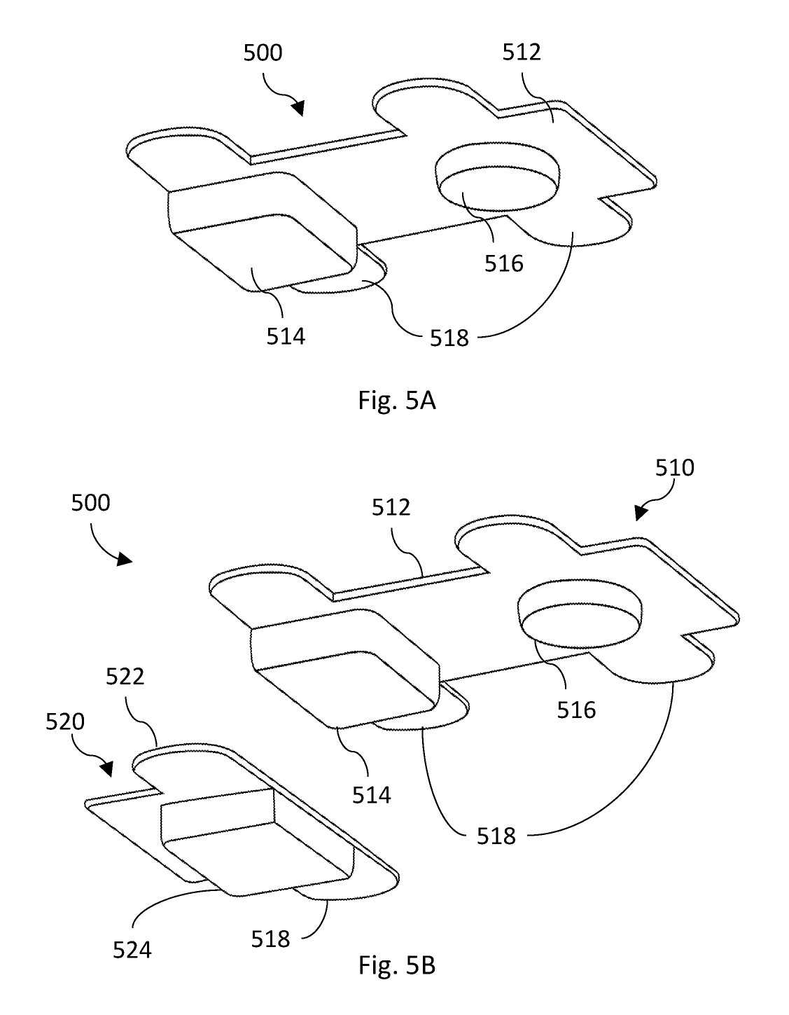

FIG. 5A is a diagram illustrating an exemplary hemostatic patch 500 in accordance with certain aspects of the present disclosure. Hemostatic patch 500 can be configured to aid in achieving hemostasis, for example, after a hemodialysis session. Hemostatic patch 500 can optionally be configured to release one or more antimicrobial agents in a way that modifies the formation of the scab beneath the patch, for example, by releasing antimicrobial agent(s) into the forming scab. Such a configuration can assist in the prevention of infection at a needling site as the modified scab not only seals the site, but also contains antimicrobial agents to kill microorganisms that may reside on and/or in the scab. A scab modified in this manner decreases a risk of infection that may occur if, in a subsequent needling session, a small piece of the scab is inadvertently pushed into the body.

While the term “hemostatic patch” is utilized herein, such is merely a convenient term evidencing one of the main goals of such a patch—the term is not intended to be limiting. For example, the hemostatic patches described herein may have additional functions and, in certain circumstances, may or may not actually assist in achieving hemostasis.

Some implementations of hemostatic patch 500 include a support layer 512 and a patient adhesive on at least a portion of the support layer. For example, a hypoallergenic adhesive may be included on the periphery of support layer 512 in order to adhere the hemostatic patch 500 to a patient's skin.

Support layer 512 may be made of, for example, a single material or a blend of natural or synthetic materials such as cotton, nylon, spandex, etc. Support layer 512 may optionally be configured to be waterproof or water-resistant.

Hemostatic patch 500 may be formed in essentially any shape and size large enough to cover the relevant area around a skin puncture site. Particular designs can include a rectangular patch with a length in the range of 1 cm to 15 cm and a width of 1 cm to 8 cm, a circular patch having a diameter in the range of 1 cm to 10 cm, or an oval patch with a long diameter range of 1 cm to 15 cm and a short diameter range of 1 cm to 8 cm. Depending on the embodiment details, hemostatic patch 500 can cover an area ranging from 1 cm² to 94 cm².

In some embodiments, as shown in FIG. 5A, hemostatic patch 500 can include one or more wings 518 (e.g., extending from support layer 512, with a length in the range of 0.1 cm to 15 cm). Such wings can be configured to include a patient adhesive and to aid in retaining hemostatic patch 500 in the desired location on a patient's skin.

As noted above with respect to the disinfecting patch and the scab removal patch, in some implementations, hemostatic patch 500 may also include a similar handle portion for easy one-handed removal.

Hemostatic patch 500 may also include a sponge 514 included on at least a portion of support layer 512, as shown in FIG. 5A. Sponge 514 may be, for example, rectangular, circular, or oval in cross section, with an area ranging from, e.g., 0.25 cm² to 80 cm². Sponge 514 can be configured to release an antimicrobial agent. For example, sponge 514 can be an antimicrobial pad or can be a pad containing an antimicrobial agent. Sponge 514 can be made of essentially any single material or blend of absorbent material(s), such as cotton, cellulose, hydrogels, etc. The sponge material may be or include one or more hydrocolloid materials such as gelatin, carboxy-methylcellulose and other polymers. Sponge 514 can be configured to be non-adherent to the wound.

Anti-microbial agents utilized with sponge 514 can include alcohols (e.g. ethanol, isopropanol), aldehydes (e.g. glutaraldehyde, formaldehyde), anilides (e.g. triclocarban), biguanides (e.g. chlorhexidine, alexidine), bisphenols (e.g. triclosan, hexachlorophene), diamidines (e.g. propamidines, dibromopropamidines), halogen-releasing agents (e.g. chlorine compounds, iodine compounds), halophenols (e.g. chloroxylenol), heavy metal derivatives (e.g. silver compounds, mercury compounds), peroxygens (e.g. hydrogen peroxide, ozone, peracetic acid), phenols and cresols (e.g. phenol, cresol) and quaternary ammonium compounds (e.g. cetrimide, benzalkonium chloride), or a topical antibiotic such as mupirocin and polysporin.

In some implementations, sponge 514 can include anti-microbial agents in solution with water. Such implementations can allow for moist healing at the wound site. Sponge 514 can be configured to maintain the solution at a certain moisture level during the wound healing process to reduce scab formation, which can be beneficial for patients undergoing repeated needling at the same site, as may occur in hemodialysis.

In some implementations, hemostatic patch 500 can be configured to provide for passive compression to aid in hemostasis. As used herein, “passive compression” means compression that is not due to an external pressure (e.g., a user pressing on a portion of hemostatic patch 500). In one exemplary implementation, sponge 514 included on support layer 512 can be configured to expand upon absorption of a fluid. In addition, support layer 512 can be configured to be substantially inelastic. As a result of these features, hemostatic patch 500 will be configured in a manner that can result in the exertion of a hemostatic force (specifically, a passive compression) upon a wound when the sponge 514 absorbs a fluid (e.g., blood).

Certain implementations of the hemostatic patch described above may also incorporate a “vessel-focused” hemostatic material included on the support layer, away from the sponge. FIG. 5A depicts an exemplary vessel-focused hemostatic material 516 which, in this case, is an additional sponge 516.

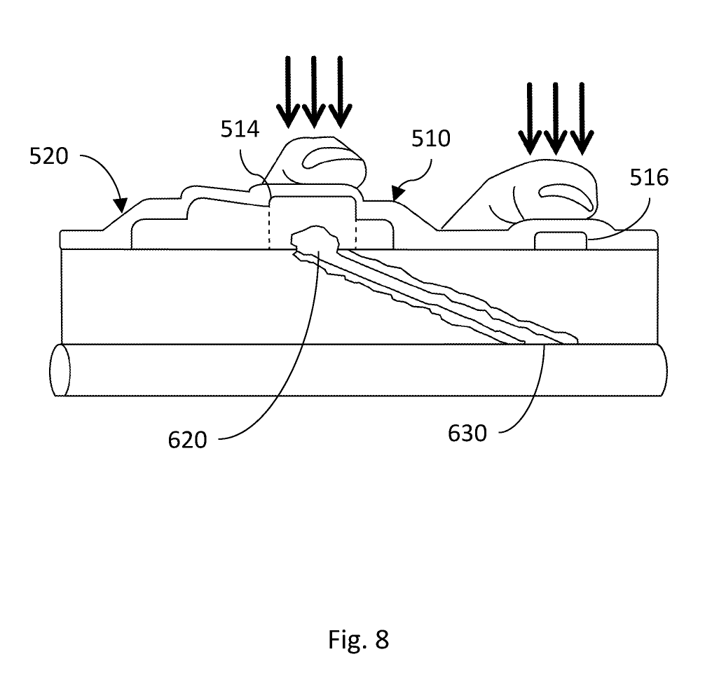

Reference to FIG. 8 can help illustrate why element 516 is referred to as a “vessel-focused hemostatic material.” Specifically, FIG. 8 shows sponge 514 of hemostatic patch 500 assisting with hemostasis at a skin puncture site 620, while the “vessel-focused” sponge 516 is assisting with hemostasis at a vessel puncture site 630, below the skin.

In the example of FIG. 8, hemostasis can be facilitated by the external application of pressure (e.g., by a hand of the patient) above skin puncture site 620 and also above vessel puncture site 630. As used herein, the term “vessel-focused” means that the sponge, material, marking, etc., is directed to applying pressure and/or facilitating hemostasis at the site of a blood vessel puncture generally below the vessel-focused material.

While the example of FIG. 8 demonstrates active compression on the vessel-focused material 516, it is contemplated that hemostatic patch 500 may be configured for passive compression at the vessel-focused material location. In one example, such may be effected through means similar to those described above with support layer 512 designed to be substantially inelastic, and vessel-focused sponge 516 configured to expand (e.g., following physical compression, rather than fluid absorption). In this way, hemostatic compression(s) may begin at one or both of the vessel puncture site and the skin puncture site following application of hemostatic patch 500—even prior to a patient or caregiver applying active compression.

The vessel-focused hemostatic material 516 may be an additional sponge and may optionally include one or more hydrocolloid materials such as gelatin, carboxy-methylcellulose and other polymers. Exemplary vessel-focused hemostatic materials can be rectangular, circular, or oval in cross section, with an area ranging from, for example, 0.25 cm² to 80 cm². The vessel-focused hemostatic material 516 may be made of an absorbent (or elastic) material essentially identical or similar to that of sponge 514, or it can be made of a non-absorbent material.

The vessel-focused hemostatic material 516 may merely be a marking on support layer 512, indicating a location where hemostatic pressure is to be applied. The marking can be a color, pattern, text, picture, or other such indication that can be visually detected by a user (e.g., the patient) to identify the location. In other implementations, vessel-focused hemostatic material 516 may simply be a portion of support layer 512 having an increased or decreased thickness compared to an adjacent portion of support layer 512 or may be anything that creates an indentation or a raised area that can be tactilely detected by a user.

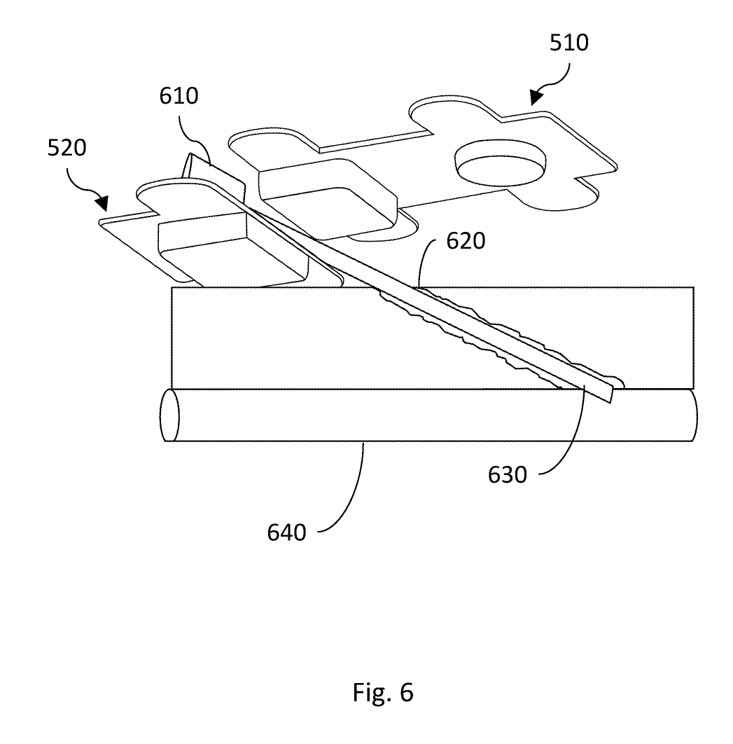

Hemostatic patches that include vessel-focused hemostatic materials can be configured for situations where, for example, a vessel-seeking needle is inserted into a patient at an angle, so that the vessel puncture site will not be directly below the skin puncture site (for example, as shown in FIG. 6). In such situations, vessel-focused hemostatic material 516 can be included on support layer 512, away from sponge 514, and generally placed at a position that will result in the application of pressure over a location corresponding to the site of a needle puncture into a vessel wall. The vessel-focused hemostatic material may therefore be placed away from sponge 514 an expected distance between the needle/skin entry point and the needle/vessel entry point, as may be predicted in a hemodialysis procedure. Depending upon the procedure, the distance between sponge 514 and vessel-focused hemodialysis material 516 may be in the range from 10 mm to 100 mm.

In some implementations of hemostatic patch 500, the distance between the sponge and the vessel-focused hemodialysis material can be designed to have dimensions correlated with an expected, estimated, or actual distance between the skin puncture site and the vessel puncture site as established by a subcutaneously implanted needle guiding device. Examples of such subcutaneous needle guiding devices are described in published International Patent Applications WO 2014/017986 and WO 2016/111650, the contents of which are incorporated by reference for their disclosure of needle guiding devices and their use. In one example, a subcutaneous needling guide be implanted such that an upper surface thereof resides at an expected or known distance (e.g., 1-2 mm) below the skin surface. A lower surface thereof can reside at an expected distance above a target blood vessel (e.g., 2-5 mm). The subcutaneous needle guide device can have a channel that guides a needle toward the target blood vessel along an intended trajectory at a predetermined angle (e.g., 30 degrees relative to a horizontal reference plane). An expected distance between a skin puncture site and a vessel puncture site (e.g., a lateral or horizontal distance) can then be determined or calculated. Sponge 514 and vessel-focused hemostatic material 516 can then be designed to have center points spaced apart by this determined distance.

Examples of hemostatic patches discussed to this point comprise a single continuous patch, as depicted in FIG. 5A. The use of such patches described above generally includes placement of the patch (and specifically sponge 514) over a needle puncture site in a patient's skin. In one particular use, the patch can be adhered in place on the patient's skin before needle removal. In this way, hemostatic patch 500 and its sponge 514 will already be in place above the needle to facilitate hemostasis as quickly as possible when the needle is removed.

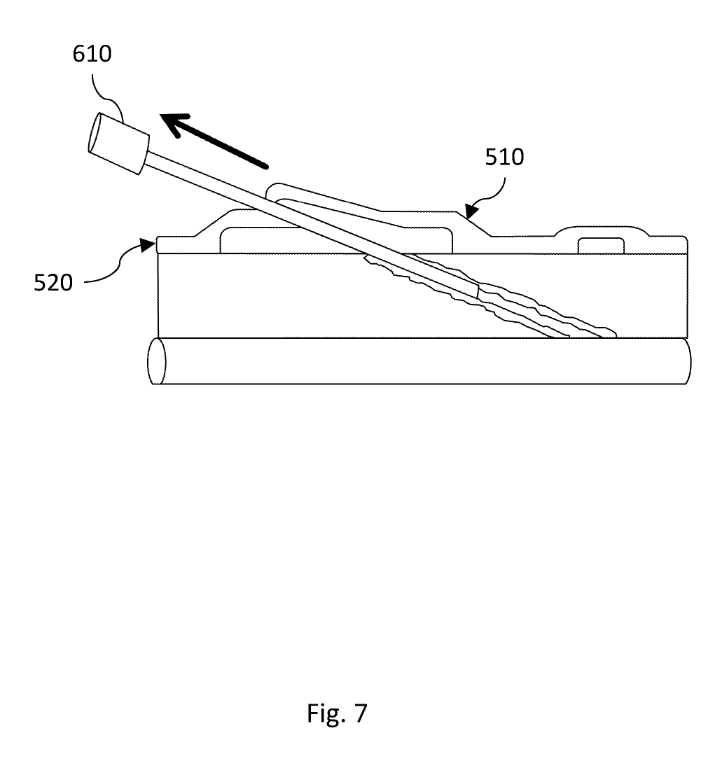

In other embodiments, an example of which is depicted in FIG. 5B, the hemostatic patch 500 may include an above-needle portion 510, and a below-needle portion 520. As shown in FIG. 6, the below-needle portion 520 of hemostatic patch 500 may be adhered to the patient a location below the needle. Subsequently, the above-needle portion 510 of hemostatic patch 500 may be adhered above the needle. At times, the above-needle portion 510 may be placed in a manner that overlaps with below-needle portion 520, as depicted in FIG. 7.

The above-needle portion 510 of hemostatic patch 500 may be configured similarly to the previously described embodiments of hemostatic patch 500 and include a support layer, a patient adhesive on a portion of the support layer, a sponge, and, optionally, a vessel-focused hemostatic material away from the sponge.

Similarly, below-needle portion 520 of hemostatic patch 500 may include a separate/second support layer, patient adhesive, and a sponge 524, as depicted in FIG. 5B. The below-needle portion 520 may also optionally include wings 518.

Either or both of the above-needle portion 510 and below-needle portion 520 of hemostatic patch 500 may include any of the features described above with regard to the single/continuous version of the hemostatic patch, for example, including a patient adhesive thereon and/or handle portion(s). Similarly, hemostatic patch 500, above-needle portion 510, and below-needle portion 520 may be designed to incorporate features and characteristics of the healing patch described in more detail below.

Either or both of the sponges 514 and 524 may also include any of the features previously described with regard to sponge 514. For example, either or both of the sponges 514 and 524 may be made of similar materials and configured to release antimicrobial agents and/or to provide for moist healing.

In some variations, above-needle portion 510 and below-needle portion 520 can be provided as a single unit that may be separated along a perforation, for example, similar to the embodiment described above in FIG. 1 with regard to the combined disinfecting patch and scab removal patch.

Overlap of above-needle portion 510 and below-needle portion 520 can act to stabilize a needle therebetween, as can be understood from FIG. 7. When a hemostatic patch 500 includes such needle-stabilizing properties, it may be beneficial to utilize the patch early in a hemodialysis procedure (instead of at, or just before, needle removal at the end of the procedure). For example, a below-needle portion 520 of hemostatic patch 500 may be put into place and adhered to the patient's skin immediately following insertion of the needle 610 through the patient's skin and into a blood vessel. Then, above-needle portion 510 may be adhered above the needle 610, resulting in at least some needle stabilization, as depicted in FIG. 7.

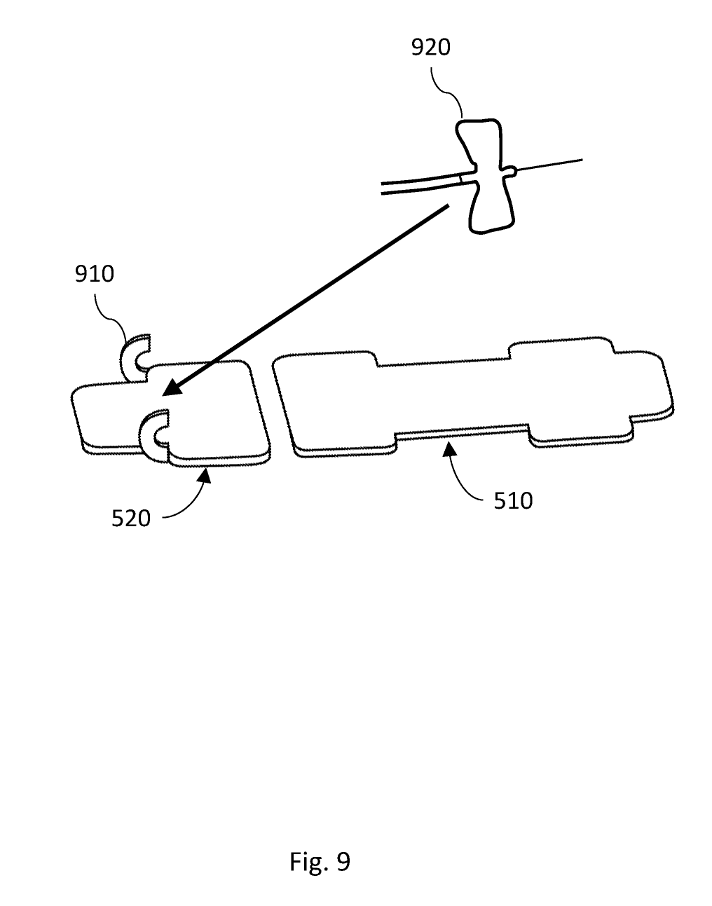

Optionally, hemostatic patch 500, or a portion of the patch, may include additional needle stabilizing elements. In one example, depicted in FIG. 9, below-needle portion 520 may include hooks 910 that can be configured to engage, for example, a butterfly needle 920 in order to reduce the likelihood that the needle will be pulled out of the needle tract in the patient.

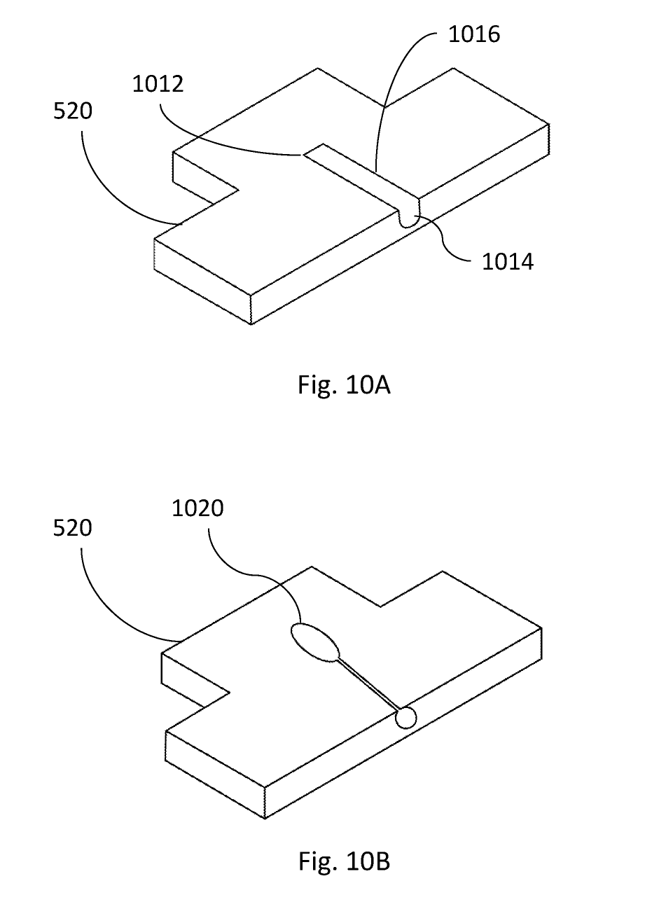

In other implementations, depicted in FIGS. 10A and 10B, the needle stabilizing element can be a slot in below-needle portion 520 that further secures needle 610. The slot can have a first end 1012, a second end 1014, and slot channel 1016 shaped to receive a needle. The slot channel can be formed to be at an angle that supports needle 610 at the preferred angle of incidence into the patient, for example, the same angle provided by a channel in a subcutaneous needle guiding device. In the embodiment of FIG. 10B, slot 1020 can be configured to substantially surround needle 610 and thereby provide improved stabilization.

An adhesive may also be utilized to act as a needle stabilizing element, for example, being included within a slot, or between below-needle portion 520 and above-needle portion 510 of hemostatic patch 500.

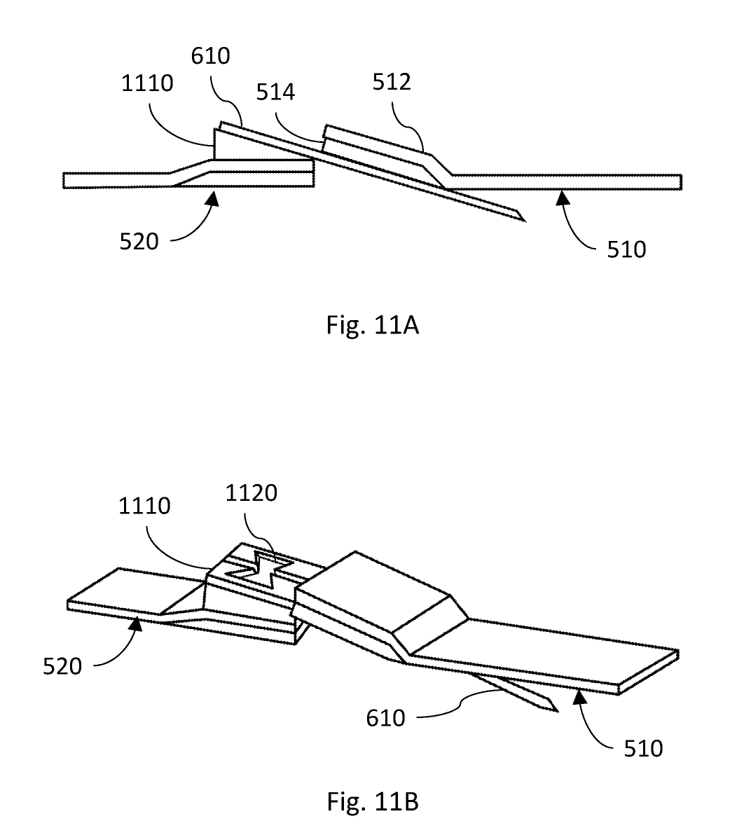

In other implementations, the needle stabilizing element can be a cutout 1120, for example, shaped to receive a butterfly needle (as depicted in FIG. 11B). It is contemplated that the stabilization element may be shaped in any way necessary to facilitate stabilizing the needle or any handle or attachment associated with the needle.

Needle stabilizing elements, as discussed above, may be included as part of hemostatic patch 500. Alternatively, an additional “wedge” or block may be utilized in with the hemostatic patch and the wedge itself may include needle stabilizing elements. FIGS. 11A and 11B depict such an exemplary wedge 1110 mounted on top of the below-needle portion 520 of hemostatic patch 500. Such a wedge may include any of the applicable needle stabilizing elements described above with regard to the hemostatic patch.

Such wedges are optionally used with the hemostatic patch and can be provided with the patch, or separately, and can optionally be configured to be removably attachable to the hemostatic patch. In one example, the bottom of a wedge may include an adhesive for securing to the top of hemostatic patch 500, or to the below-needle portion 520 of hemostatic patch 500. In another example, the wedge and hemostatic patch 500 may be configured to snap together.

In some implementations, wedges may be designed to have a top surface with an angle corresponding to an expected needle angle. In this manner, the wedge may provide further support for the needle at its preferred angle of incidence into the patient, for example, the same angle provided by a channel in a subcutaneous needle guiding device. It is contemplated that this angular support feature may similarly be provided in the design of the hemostatic patch 500 itself (for example, including such an angle in the design of below-needle portion 520, instead of in a separate wedge).

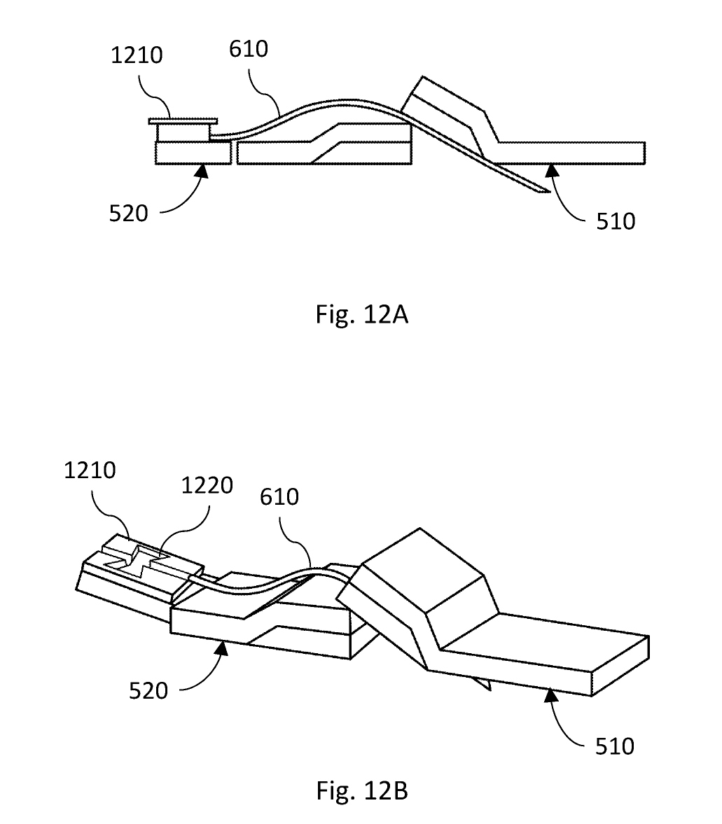

In certain implementations, a block or wedge, similar to those described above, may instead be utilized in location adjacent to or behind the hemostatic patch (as depicted in FIGS. 12A and 12B). In such implementations, it is contemplated that either the wedge or the hemostatic patch, or both of them, may include needle stabilizing elements as described herein.

Some implementations of the needle stabilizing elements described herein (including the wedges), may be made of materials such as, but not limited to, metals (for example, stainless steel, titanium, copper, silver, gold, platinum, surgical steel, cobalt, chromium, nickel, molybdenum, tungsten, tungsten carbide, alloys of these metals and so on), plastics or polymers (for example, acrylic, polyethylene, high density polyethylene, low density polyethylene, polyurethane, polyamide, polyethylene terephthalate, polyvinyl chloride, polystyrene, polycarbonate, other polyesters, polypropylene, polyether ether ketone, and so on), ceramics, glass, etc.

While the term “needle” stabilizing element is used herein, is contemplated that the stabilizing element described may be configured to stabilize other instruments, for example, a cannula that may be used in hemodialysis. Implementations of the current subject matter may include any number or combination of needle stabilizing elements disclosed herein to cause a needle, tube, or the like, to remain in place during a medical procedure.

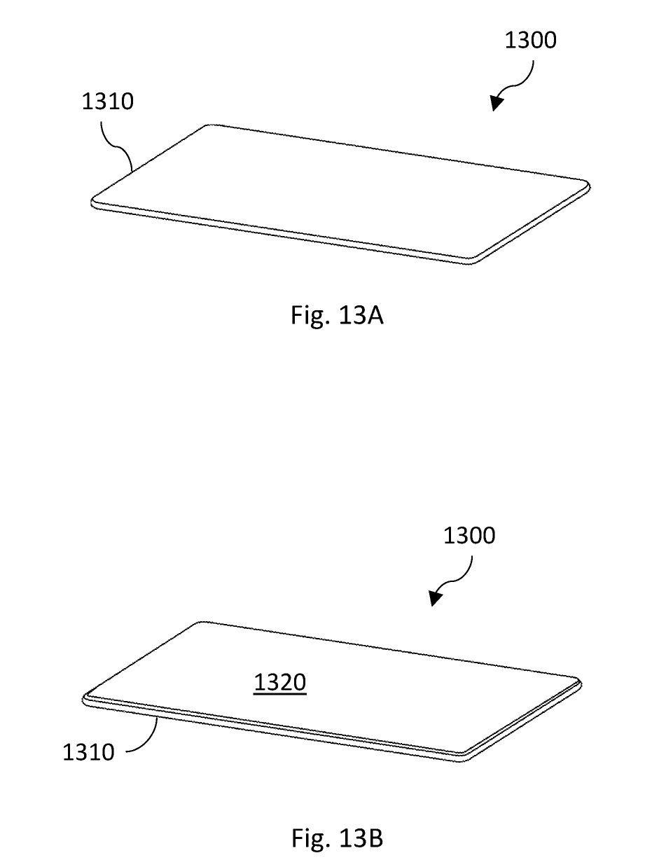

FIGS. 13A and 13B are diagrams illustrating top and bottom views of an exemplary healing patch 1300 in accordance with certain aspects of the present disclosure. Healing patch 1300 may be used to promote healing and/or to release antimicrobial agents at a skin puncture site. Healing patch 1300 may be constructed of a material with a coating, or may be designed to include a liquid suspended in the material itself. In other implementations, as shown in FIG. 13, healing patch 1300 can be constructed as multiple layers where first layer 1310 can be, for example, a generally non-absorbent material and second layer 1320 can abut first layer 1310 and can contain a liquid that can be released onto the skin puncture site to make or keep it moist and/or to release antimicrobial agents.

Some implementations of the patches described herein may include sensors, which, in certain embodiments, can establish communication with a network, computer server, and/or other devices utilizing, for example, a microprocessor. Other implementations can include, additionally or alternatively, one or more chemical reactive components. These particular embodiments may, for example, enable the reading of vital signs or other patient characteristics automatically, upon request by a user, or upon receipt of a command from another device. With the ability to read such information, relevant services and medical attention can be delivered to the patient. Chemical reactive component(s) and/or electrical sensors can also serve as visual indicators or can generate data to aid in determining appropriate steps to be taken by a patient or caregiver.

Some implementations of the patches described herein may include chemical reactive components configured to react with particular markers present in the blood to provide information. Some such markers may include, but are not limited to, Hba1c, cholesterol, triglycerides, red blood cells, white blood cells, platelets, fibrin, fibrinogen, other blood clotting cascade molecules, etc. Chemical reactive components can also, for example, detect the presence of bacteria at the site of needle injury, monitor the amount of antimicrobial agents released, detect the moisture level of the site, the extent of scab formation, the extent of healing, and so on. Information can be communicated as data through microprocessors and transmitters or can be evidenced by a detectable change in the hemostatic patch. Detection mechanisms can include, but are not limited to, colorimetric, fluorescent, luminescent, and so on. Colorimetric changes in chemical reactive components may be detected with the naked eye. Fluorescence or luminescent changes may be detected with a fluorescence/luminescence detector for higher sensitivity.

The hemostatic patch can also act as a blood sampling/collection platform for use with one or more other types of blood marker detection devices. For example, the hemostatic patch can collect blood that can be used by a glucose monitor for glucose level detection.

Electrical sensing components can be embedded in any of the patches described herein to enable detection of particular types of signals. Such electrical sensing components may include, but are not limited to, thermoelectric components, piezoelectric materials, induction sensors, impedance sensors, thermocouples for temperature sensing, and so on. Electrical sensing components may be powered by a thin film battery or a thermoelectric material that converts body heat into electrical energy. A piezoelectric material can also be used for short term powering of an active sensor. For example, by pressing on the piezoelectric material, the mechanical deformation can be converted to electricity that may be sufficient to power the transmission of signals to a network or other computing system. The electrical sensing components can be configured to detect, sense, or measure parameters such as, but not limited to, skin temperature, skin moisture level, extent of bleeding or blood clotting, extent of scab formation, extent of wound healing, electrolyte concentration, needle dislodgement, etc.

In one instance, a thermocouple can be incorporated into or onto a patch and combined with microprocessor and a transmitter to interface with a network to allow a computing device (e.g., a smartphone, computer display, etc.) to display the localized skin temperature (e.g., at a site of dialysis cannulation). The patch may also be configured to allow for the display of information on the patch itself. This can be a useful form of early diagnosis of localized site infection marked by an elevated temperature. The thermocouple may be powered by either a thin film battery or a thermoelectric material.

Impedance detection can detect an impedance change in the area of skin around a skin puncture site, with or without the help of an external circuit. For example, such can be used to detect needle dislodgement in response to a change in the position of a stainless steel needle during dialysis, which can correspondingly cause a change the impedance of a circuit present on the patch that is present around the needle. Impedance tracking can also be performed on a healing patch to detect or estimate the moisture level of the healing patch, the extent of scab formation and/or the healing of the skin underneath.

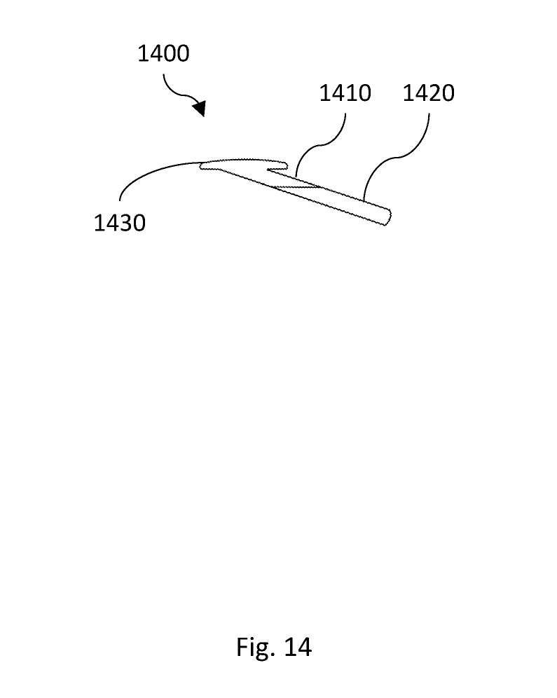

FIG. 14 is a diagram illustrating an exemplary wound closure apparatus 1400 (which may also be referred to herein as an “artificial scab”) in accordance with certain aspects of the present disclosure. The exemplary artificial scabs disclosed herein may be designed to, among other things, close, seal, or plug at least the upper portions of a needle tract (e.g., a tract created by way of the buttonhole technique that may be employed in hemodialysis). These artificial scabs may also be designed to reduce the risk of infection of a tract by providing and/or releasing antimicrobial agents, for example, between successive dialysis sessions.

An artificial scab 1400, as contemplated herein, may contain a head portion 1410 and a rod portion 1420, extending from the head portion, as depicted in FIG. 14. The head 1410 may refer only to a top portion 1430 of the apparatus, or head 1410 may also incorporate a portion of the artificial scab extending away from top (e.g., in the direction of rod 1420), as shown in the example of FIG. 14.

Rod 1420 may be made of natural or synthetic, polymeric or metallic materials, such as poly(lactic acid), polycaprolactone, silver, titanium, gold, and so on. Depending upon the embodiment details, rod 1420 can be made with a diameter ranging from 0.5 mm to 10 mm and with a length ranging from 3 mm to 30 mm. Some implementations can include rod 1420 being connected to head 1410 at an oblique angle, as illustrated in FIG. 14. Examples of oblique angles can include approximately 20, 30, 45, or 60 degrees from the horizontal illustrated in FIG. 14. However, other implementations may have rod 1420 at an approximately 90 degree angle with the horizontal (i.e., not oblique).

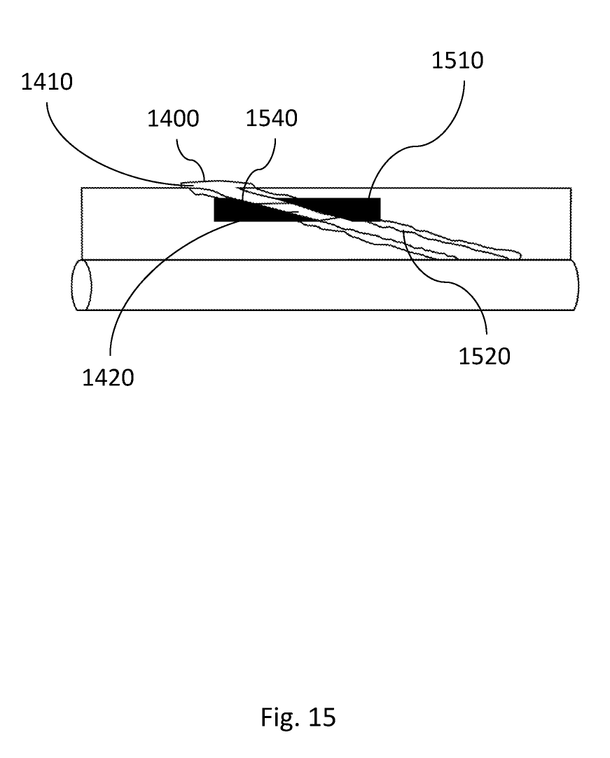

FIG. 15 is a diagram illustrating an exemplary artificial scab 1400 engaging a subcutaneous needling guide 1510 in accordance with certain aspects of the present disclosure. In situations where a patient has a subcutaneous needling guide 1510 implanted, insertion of rod 1420 into needle tract 1520 can result in portions of rod 1420 entering into a channel 1540 of subcutaneous needle guide 1510. In various embodiments, artificial scab 1400 can thus be intentionally angled (e.g., having the corresponding oblique angle) to enable rod 1420 to at least partially extend into the channel of subcutaneous needling guide 1510. The artificial scab 1400 can also be configured to have a shape and cross-sectional area or diameter that fits within or mates with channel 1540 of the subcutaneous needle guide 1510. In some implementations, rod 1420 may be configured to provide for an interference fit with the subcutaneous needling guide 1510.

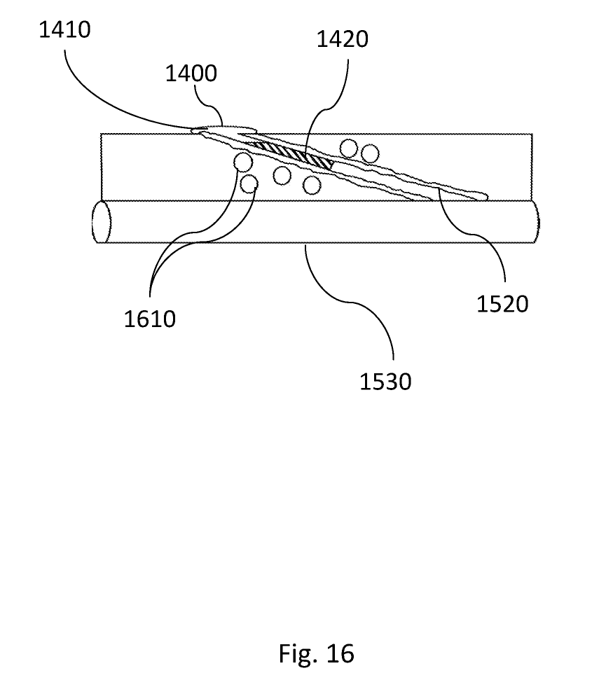

FIG. 16 is a diagram illustrating an exemplary artificial scab 1400 constructed of two different materials in accordance with certain aspects of the present disclosure. Implementations of artificial scab 1400 can include, for example, a head portion 1410 constructed from a first material and a rod portion 1420 constructed from a second material that is different from the first material.

In some implementations, rod 1420 may include silver and may be inserted into needle tract 1520 to reside for a limited period of time, for instance, between successive hemodialysis sessions. Rod 1420 may provide for and/or release one or more types of antimicrobial agents, such as silver ions 1610, into needle tract 1520, in addition to maintaining the tract's patency between dialysis sessions.

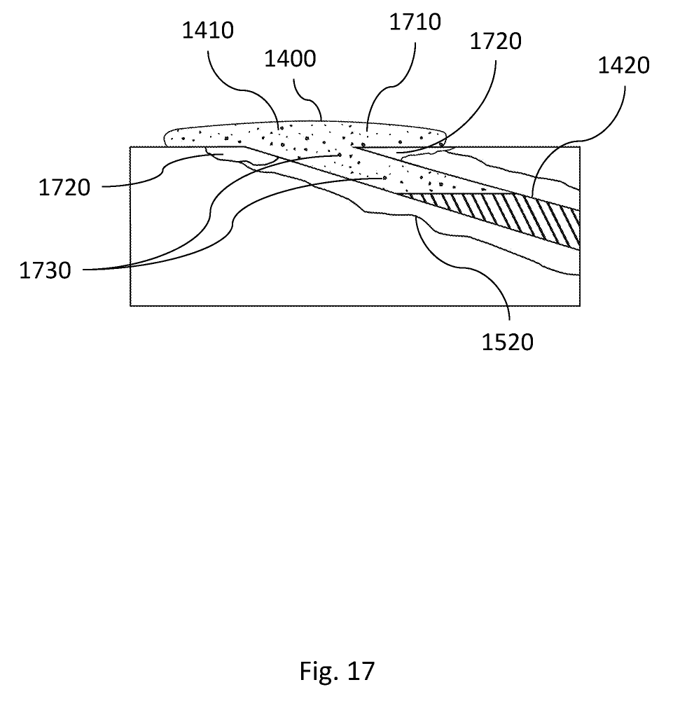

FIG. 17 is a diagram illustrating an exemplary artificial scab 1400 with a porous structure 1710 in accordance with certain aspects of the present disclosure. In some embodiments, head 1410 of artificial scab 1400 may be only partially porous, including porosity on a wound-facing portion in order to facilitate formation of a scab adhering the apparatus to a wound.

In some implementations, porous structure 1710 can include calcium phosphate, or be a calcium phosphate-alginate blend, similar to that found in bone cements. Porous structure 1710 can be further configured to release an antimicrobial agent into its surrounding area, including into a natural scab 1720 that may form between a patient's skin and the artificial scab 1400 itself. As such, in some implementations, structure 1710 can include an antimicrobial agent inside pores 1730 of the porous structure 1710.

The antimicrobial agent(s) released can include one or more antiseptic agents such as alcohols (e.g. ethanol, isopropanol), aldehydes (e.g. glutaraldehyde, formaldehyde), anilides (e.g. triclocarban), biguanides (e.g. chlorhexidine, alexidine), bisphenols (e.g. triclosan, hexachlorophene), diamidines (e.g. propamidines, dibromopropamidines), halogen-releasing agents (e.g. chlorine compounds, iodine compounds), halophenols (e.g. chloroxylenol), heavy metal derivatives (e.g. silver compounds, mercury compounds), peroxygens (e.g. hydrogen peroxide, ozone, peracetic acid), phenols and cresols (e.g. phenol, cresol) and quaternary ammonium compounds (e.g. cetrimide, benzalkonium chloride), or a topical antibiotic such as mupirocin and polysporin. Alginate solution can be added to the calcium phosphate in a formulation of 2% to 4% alginate in a 5% to 9% sodium hydrogen phosphate solution. The porosity of head 1410 allows the natural scab formed to be strongly adhered to head 1410, such that upon removal of artificial scab 1400, the natural scab lining will be removed along with the removal of artificial scab 1400.

Artificial scab 1400 can be deployed after a dialysis session (e.g., after use of a hemostatic patch 500) to close or to seal a wound, in place of a natural scab. This can provide a wound plug that, upon its insertion into the needle tract 1520, resides in at least the outer portions of the tract. A silver-containing portion of artificial scab 1400 can release silver ions 1610 into the surrounding tissue after it is deployed to prevent infection. Artificial scab 1400 may also be configured to release other antimicrobial materials, substances, compositions, or compounds into needle tract 1520.

Head 1410 of artificial scab 1400 can be adhered to the skin via formation of a natural scab 1720. Antimicrobial agents may be released from head 1410 to keep the natural scab 1720 free from infectious agent(s). During later dialysis sessions, once artificial scab 1400 is removed, at least the portion of the needle tract 1520 in which rod 1420 was disposed can be open for easy additional needling.

An exemplary method for use of an artificial scab 1400 may include removing an instrument from a tract in a patient (i.e., the tract extending through and below skin of the patient), and then inserting a rod portion of the artificial scab into the tract until a head portion of the apparatus makes contact with the skin of the patient (the instrument being removed from the tract in the patient may be a needle used in hemodialysis). In other exemplary methods, a portion of the tract may pass through a subcutaneous needle guide, and insertion of the rod portion of the artificial scab can include insertion into a channel of the subcutaneous needling guide. In certain implementations, insertion of the rod portion into the subcutaneous needling guide may require the application of pressure to ensure an interference fit between the rod portion and the channel of the subcutaneous needling guide.

Various embodiments of technologies disclosed herein may be utilized in medical care, and specifically in hemodialysis. Exemplary uses of these technologies are described below but are not intended to be limiting. The technologies of the present disclosure may also be utilized in alternative ways.

Prior to a hemodialysis session, a patient may utilize a disinfecting patch consistent with the present disclosure to cleanse the area where needling will take place. In one particular aspect, the disinfecting patch may be applied by the patient, with one hand, and adhered to the patient's skin. The disinfecting patch can be left on for a particular specified duration that can depend on the type of disinfectant material used. Exemplary specified durations may range from, for example, 15 seconds to 15 minutes. After wearing the disinfecting patch for the specified duration, the patch may then be peeled off by the patient with one hand (preferably by way of a handle, as disclosed herein), and discarded.

In certain hemodialysis methods, particular needling sites are utilized repeatedly. With these methods, scabs from prior sessions must be removed. A scab removal patch, as described herein, may be applied by the patient, with one hand, and adhered to the patient's skin over the site of the scab. The scab removal patch may soften the scab and adhere to the scab, preferably with maximum contact between the scab removal patch's adhesive and the scab itself. The scab removal patch may be worn for a specified duration, for example 10 minutes, and then peeled off by the patient with one hand (preferably by way of a handle, as disclosed herein), and discarded. The scab removal patches of the present disclosure can facilitate maximum scab material removal and thereby decrease the possibility of scab particles (which may contain bacteria) being pushed into the body during needling.

After disinfection and scab removal, needling and hemodialysis may begin. In one implementation of the technologies disclosed herein, a wedge or a below-needle portion of a hemodialysis patch (or both) may be put in place adjacent a needling site prior to needling, or just after needle insertion. Such instruments may include needle stabilization elements that can help keep a needle in place during the dialysis session.

At the end of the hemodialysis session, a hemostatic patch, as described herein, may be applied with one hand by the patient. In one implementation, the patient would first adhere a below-needle portion of the hemostatic patch just below the needle, which is still in place within the patient. The patient would then adhere an above-needle portion of the hemostatic patch in a manner that would result in an overlap between the two portions of the patch, with the needle in-between. After both portions of the hemostatic patch are in place, the needle can be removed. The subsequent absorption of blood into the sponge(s) of the hemostatic patch can result in a passive, compressive hemostatic force being applied to the needling site, especially when a support layer of the hemostatic patch is configured to be substantially inelastic.

In certain embodiments, the hemostatic patch can include a vessel-focused hemostatic material positioned over the point where the needle punctures the blood vessel. The patch can be further configured to apply a passive hemostatic force at this location, as described herein. While passive hemostatic forces can be created by the patch designs, a patient or caregiver can also actively apply pressure to the hemostatic patch at the needle entry point on the skin and also above the needle entry point into the blood vessel. In situations where the patch is used on a patient having a subcutaneously implanted needle guiding device with upper and/or lower surfaces that are planar or which have an overall surface radius of curvature that is significantly larger than that of the target vessel above which the subcutaneous needle guiding device resides, such device surfaces can aid the distribution and effectiveness of the compressive hemostatic pressures or forces applied by the patch or by the patient/caregiver's fingers.

In certain exemplary methods, the hemostatic patch may release antimicrobial agent(s) and/or include liquids to help achieve moist healing. Such hemostatic patches may be removed, for example, between 6 and 48 hours after application. In other methods, the hemostatic patch may be removed shortly after hemostasis is achieved and a separate healing patch may be applied. Exemplary healing patches may include antimicrobial agents and may also be hydrocolloidal for moist healing and scab minimization.

In certain methods, an artificial scab can be deployed after a dialysis session (e.g., after use of a hemostatic patch) to close or seal a wound, in place of a natural scab. As described herein, an artificial scab may be inserted into the needle tract and may be designed to release antimicrobial elements into and around the tract. In cases where the patient has a subcutaneous needle guide implanted, insertion of the artificial scab may include insertion into the needle guide and may require the application of pressure to ensure an interference fit between the artificial scab and the channel of the needle guide.

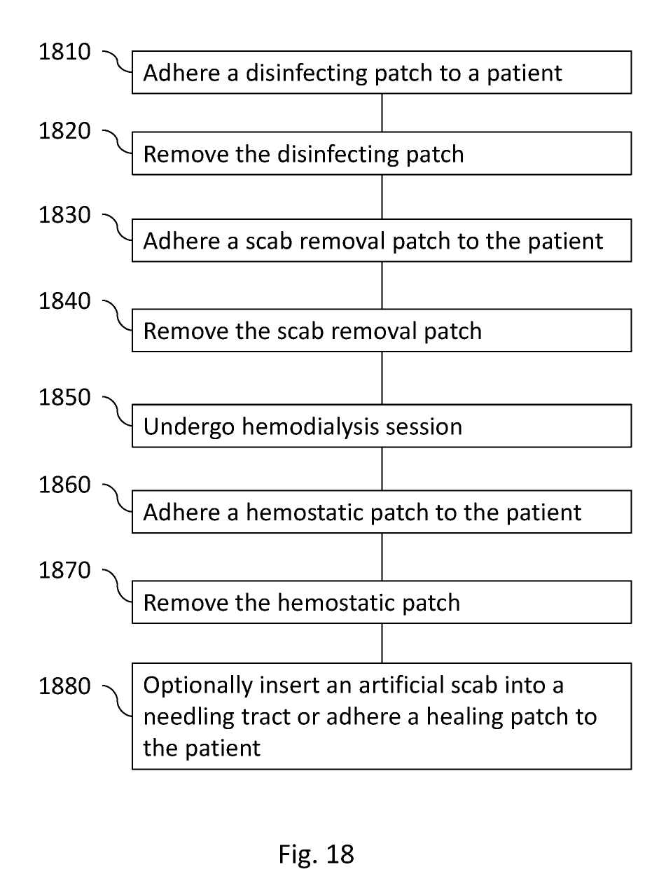

In accordance with the above disclosure, one exemplary method for use of technologies disclosed herein may thus include the following steps, as depicted in FIG. 18:

At 1810, adhere a disinfecting patch to a patient;

at 1820, remove the disinfecting patch;

at 1830, adhere a scab removal patch to the patient;

at 1840, remove the scab removal patch;

at 1850, undergo a hemodialysis session;

at 1860, adhere a hemostatic patch to the patient;

at 1870, remove the hemostatic patch; and

at 1880, optionally insert an artificial scab into a needling tract or adhere a healing patch to the patient.

In conjunction with this exemplary method, any of the technologies described herein may also be utilized (e.g., the scab removal patch may have adhesive configured to avoid tenting, etc.).

The present disclosure contemplates the provision of kits to facilitate improved hemodialysis procedures that may contain any number and combination of the apparatuses described herein. Such kits may optionally include additional items such as instructions for use, packaging, sterilization wrapping, etc.

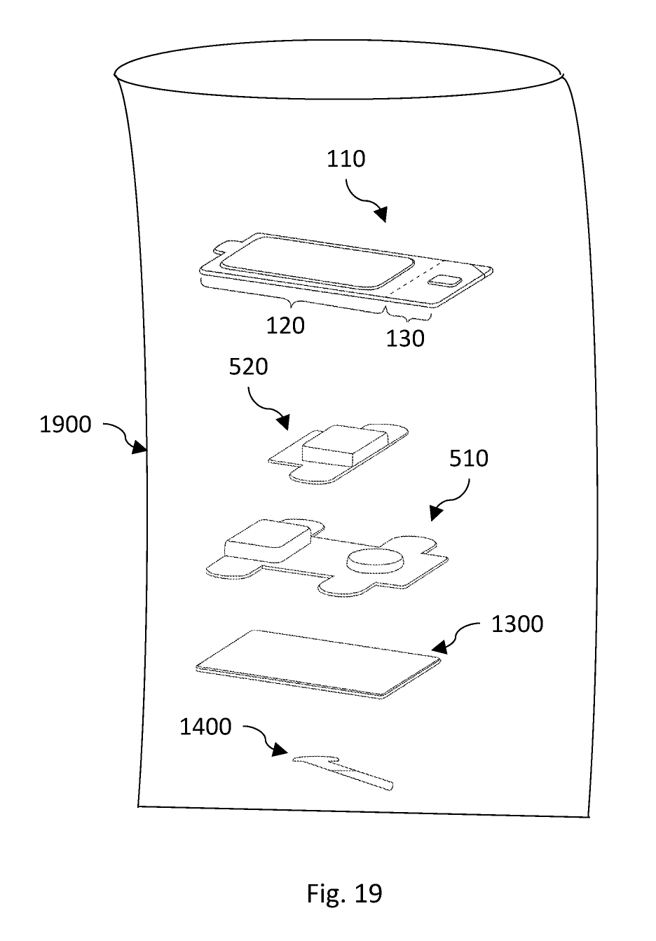

FIG. 19 is a diagram illustrating one example of a kit 1900 that may include a pre-needling patch 110 (which may be a combined disinfecting patch 120 and scab removal patch 130), a hemostatic patch (which may include an above needle portion 510 and a below-needle portion 520) and a healing patch 1300 and/or an artificial scab 1400.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, computer programs and/or articles depending on the desired configuration. Any methods or the logic flows depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

1. A hemostatic patch comprising:
a support layer;
a patient adhesive included on at least a portion of the support layer;
a sponge included on at least a portion of the support layer; and
a vessel-focused hemostatic material included on the support layer, away from the sponge.

2. The hemostatic patch of claim 1 wherein the vessel-focused hemostatic material is at a position on the support layer for applying pressure over a location corresponding to a site of a needle puncture into a vessel wall.

3. The hemostatic patch of claim 1 wherein the vessel-focused hemostatic material is an additional sponge.

4. The hemostatic patch of claim 1 wherein the vessel-focused hemostatic material is a marking on the support layer indicating a location where pressure is to be applied.

5. The hemostatic patch of claim 1 wherein the vessel-focused hemostatic material is a portion of the support layer having an increased or decreased thickness.

6. The hemostatic patch of claim 1 wherein a distance between the sponge and the vessel-focused hemostatic material is an expected distance between a needle/skin entry point and a needle/vessel entry point in a hemodialysis procedure.

7. (canceled)

8. The hemostatic patch of claim 1 further including a handle portion.

9. The hemostatic patch of claim 8 wherein the handle portion is a protruding portion of the support layer or wherein the handle portion is a peripheral portion of the support layer that is devoid of the patient adhesive.

10. (canceled)

11. The hemostatic patch of claim 1 further comprising a chemical reactive component configured to perform at least one of: detecting a presence of bacteria, monitoring an amount of antimicrobial agents released, detecting a moisture level, detecting a presence of red blood cells, white blood cells, platelets, fibrin, or fibrinogen, or wherein the hemostatic patch is further configured to perform at least one of: communicating data through a transmitter or causing a detectable change in the hemostatic patch.

12. (canceled)

13. The hemostatic patch of claim 1 further comprising an electrical sensing component configured to detect at least one of: skin temperature, skin moisture level, extent of bleeding or blood clotting, extent of scab formation, extent of wound healing, electrolyte concentration, or needle dislodgement, or wherein the hemostatic patch is further configured to perform at least one of: communicating data through a transmitter or displaying information at the hemostatic patch.

14. (canceled)

15. A hemostatic patch comprising:
an above-needle portion including
a support layer;
a patient adhesive included on at least a portion of the support layer;
a sponge included on at least a portion of the support layer; and
a vessel-focused hemostatic material included on the support layer, away from the sponge; and
a below-needle portion including
a second support layer;
a second patient adhesive included on at least a portion of the second support layer; and
a second sponge included on at least a portion of the second support layer.

16. The hemostatic patch of claim 15 wherein the below-needle portion further includes a needle stabilizing element.

17. The hemostatic patch of claim 16 wherein the needle stabilizing element comprises hooks configured to engage a butterfly needle, or wherein the needle stabilizing element is a butterfly-shaped indentation in the below-needle portion of the hemostatic patch, or wherein the needle stabilizing element is a slot within the below-needle portion of the hemostatic patch.

18. (canceled)

19. (canceled)

20. The hemostatic patch of claim 15 further comprising a wedge configured to be placed above or adjacent to the below-needle portion of the hemostatic patch.

21. The hemostatic patch of claim 20 wherein the wedge is configured to have a top surface with an angle corresponding to an expected needle angle.

22. The hemostatic patch of claim 20 wherein the wedge further includes a needle stabilizing element.

23. The hemostatic patch of claim 15 wherein the first sponge and the second sponge are configured to release antimicrobial agents.

24. (canceled)

25. (canceled)

26. (canceled)

27. A hemostatic patch comprising:
a support layer;
a patient adhesive included on at least a portion of the support layer; and
a sponge included on at least a portion of the support layer and configured to expand upon absorption of a fluid;
wherein the support layer is configured to be substantially inelastic so that a hemostatic force will be exerted upon the sponge's absorption of a fluid.

28. The hemostatic patch of claim 27, further comprising a plurality of wings extending from the support layer.

29. The hemostatic patch of claim 27, wherein the sponge is configured to release an antimicrobial agent.
